(19) United States
(12) Patent Application Publication
(10) Pub. No.: US 2004/0176612 A1
(43) Pub. Date: Sep. 9, 2004

(54) Method for preparing n-($g(v)-bromoalkyl)phthalimides

(76) Inventors: Jean-Philippe Gillet, Brignais (FR); Christophe Ruppin, Saint-Pierre D'Albigny (FR)

Correspondence Address: Marcy M Hoefling, Albemarle Corporation, 451 Florida Street, Baton Rouge, LA, 70801-1765, US

(21) Appl. No.: 10/490,886

(22) PCT Filed: Sep. 26, 2002
(86) PCT No.: PCT/FR02/03273

(30) Foreign Application Priority Data
Sep. 27, 2001 (FR) 01/12420

(51) Int. Cl. C07D 209/48

(52) U.S. Cl. 548/473

(57) ABSTRACT

The invention concerns a procedure for preparation of N-(ω-bromoalkyl)phthalimides which consists of reacting potassium phthalimide with an α,ω-dibromoalkane without cosolvent, at a temperature from 50° C. to 130° C. by using a molar ratio of α,ω-dibromoalkane to potassium phthalimide from 2.5 to 6, then of removing excess α,ω-dibromoalkane, under reduced pressure at a temperature equal to 150° C. at most, of taking up the reaction medium again with a low boiling point alcohol at a temperature near that of the boiling point of said alcohol, of filtering at this temperature, of cooling the filtrate obtained and then of recovering the N-(ω-bromoalkyl)phthalimide.

The present invention concerns a procedure for preparation of N-(ω-bromoalkyl)phthalimides with formula:

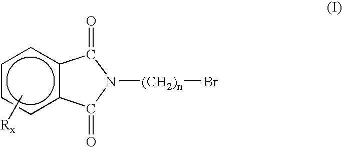

in which n is a whole number from 2 to 8, R represents a hydrocarbon radical, straight-chain or branched, with 1 to 4 carbon atoms, a halogen atom, or an alkoxy radical, x is equal to 0, 1, 2, 3 or 4 and when x=2, 3 or 4, the R radicals may be identical or different.

These compounds are intermediates in the synthesis of pharmaceutical products such as antibiotics and neuroleptics.

Numerous methods for preparing these compounds are mentioned in the literature.

Thus, in patent U.S. Pat. No. 4,705,892, a method is described to prepare N-(ω-bromoalkyl)phthalimides, especially compounds with formula (I) or [sic; where] n=5 or 6 which consists of reacting the phthalic anhydride with an α,ω-aminoalcohol in a hydrocarbon solvent such as toluene, benzene, xylene; of removing the reaction water by azeotropic distillation and then of reacting the obtained product with formula:

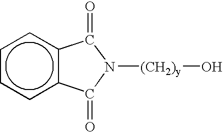

(y=5 or 6) with PBr₃ in a hydrocarbon solvent such as toluene.

The obtained products are purified by crystallization in ethanol.

This way of operating presents the drawback of using reagents (aminoalcohol) that are accessible with difficulty, of using hydrocarbon solvents and of using PBr₃ which necessarily causes expensive treatment of the effluents.

The method most currently used for preparing compounds with formula (I) consists of reacting potassium phthalimide (II) with an α,ω-dibromoalkane (III) according to reaction (1):

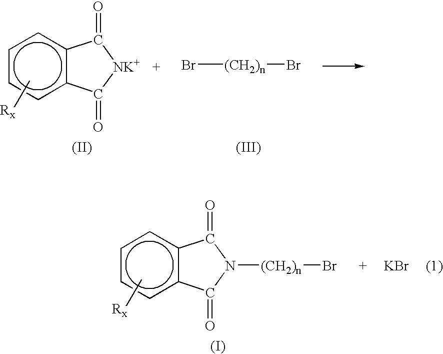

The potassium salt (III) may be used as is. It may also be prepared in situ generally by the action of potassium carbonate (anhydrous) or potash on the phthalimide with formula:

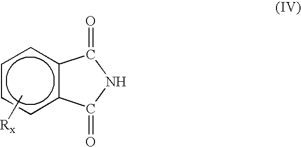

In patent U.S. Pat. No. 5,541,230, a procedure for preparation of N-(4-bromobutyl)phthalimide is described that consists of reacting 30 mmol of potassium phthalimide with 100 mmol of 1,4-dibromobutane in 50 mL of DMF. The reaction medium is stirred for 2 h at 60° C. and then the DMF and the excess 1,4-dibromobutane are evaporated under reduced pressure. The remaining residue is extracted with CHCl₃ and water. The compound obtained is crystallized with ethanol.

This procedure presents the drawback of using three solvents:

DMF for condensation,

CHCl₃ for the extraction of the product from the reaction medium and

ethanol for carrying out the purification.

In patent application EP 314 098, N-(4-bromobutyl)phthalimide is also obtained by condensation of potassium phthalimide with 1,4-dibromobutane used according to a molar ratio of dibrominated compound/potassium salt equal to 4.6 in DMF.

The reaction is carried out with stirring of the reaction medium at 90/100° C. for 10 h.

Crystals of KBr which precipitate are removed by filtration. The filtrate is concentrated under reduced pressure to remove the excess 1,4-dibromobutane and the residue is purified by a silica gel chromatography column.

This way of operating is totally unacceptable for an industrial procedure. In addition, the condensation is carried out in toxic DMF solvent.

T. A. CRABB et al. (J. Chem. Soc. Perkin Tran. I, pp 191-195 (1985)) obtained N-(4-bromobutyl)phthalimide with a yield of 32% by carrying out the condensation of potassium phthalimide with 1,4-dibromobutane used according to a molar ratio of dibrominated compound/potassium salt equal to 1.35 in acetone by heating at reflux for 24 h. The KBr is removed by filtration and the solvent is evaporated. The obtained product is purified by successive recrystallizations with petroleum ether.

In patent application EP 330 625, N-(3-bromopropyl)phthalimide is obtained with a yield of 72% according to the following method.

In a 1-L round-bottomed flask provided with stirring and cooling means are introduced 101 g (0.5 mol) of 1,3-dibromopropane, 250 mL of acetone and 15 g of potassium phthalimide. The reaction medium is brought to reflux under stirring, to which is added at intervals of 1 h, 15 g, 10 g and 6.3 g of potassium phthalimide (or a total of 46.3 g: 0.25 mol). Reflux is maintained for 24 h in all. Next KBr is removed by filtration and the acetone is evaporated.

The oil obtained is distilled under reduced pressure to remove the excess of 1,3-dibromopropane and then the N-(3-bromopropyl)phthalimide is recrystallized twice in ethanol to remove the diphthalimidopropane, product of dicondensation.

Generally, the procedures that we have just described present the drawback of using 2, even 3 solvents that are either very flammable (acetone) or toxic (DMF) and in addition, these procedures require numerous operations for purification of the products.

P. L. Salzberg and J. V. Supniewski (Organic Syntheses, Coll. Volume 1, Second edition, John Wiley and Sons Inc. 1941, New York pp. 119-121) describe the preparation of β-bromoethylphthalimide according to the following method.

Into a 1-L round-bottomed flask equipped with effective stirring and a reflux condenser, are introduced 150 g of potassium phthalimide (0.81 mol) and 450 g of 1,2-dibromoethane (2.4 mol) with a boiling temperature of 129° C./131° C.

Stirring is started up and the mixture is heated approximately 12 h by means of an oil bath kept at a temperature of 180/190° C.

Next, excess dibromoethane is removed under reduced pressure. 290 to 295 g of 1,2-dibromoethane are recovered.

The crude product is separated from the KBr by dissolving in 300 mL of 98/100% alcohol brought to reflux until the black oil is entirely solubilized.

The hot ethanolic solution is filtered; the KBr cake obtained is washed with hot ethanol. The alcohol of the ethanolic filtrate is distilled under reduced pressure and then the dry residue is treated at reflux with 500 mL of CS₂ for approximately 50 min in order to separate the soluble β-bromoethylphthalimide from the insoluble diphthalimidoethane. The suspension is hot filtered and the CS₂ is removed under reduced pressure.

The β-bromoethylphthalimide obtained with a yield of 70-80% is presented in the form of yellow-brown crystals melting at 78-80° C.

To obtain a pure product, it is necessary to recrystallize the crude product with dilute alcohol in the presence of decolorizing activated charcoal.

R. H. Mizzoni et al. (J.A.C.S. Vol. 76, page 2416, 1954) adapted this method for preparing N-(4-bromobutyl)phthalimide that they obtained with an overall yield of 61% after treatment of the mother liquors.

The drawback of the latter procedures is the use of reaction temperatures that are too high and that create by-products and colorations of desired products requiring numerous purification operations with flammable solvents (CS₂, cyclohexane) and decolorization operations.

The applicant has found a simple procedure for preparation of compounds with formula (I) which consists of condensing potassium phthalimide (II) with an α,ω-dibromoalkane (III) without use of cosolvents and then of extracting and purifying the product obtained with the minimum of operations without using toxic and/or very flammable solvents.

Therefore, the invention has as a goal a procedure for preparation of N-(ω-bromoalkyl)phthalimides with formula:

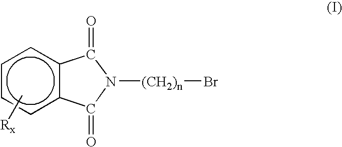

in which n is a whole number from 2 to 8, and preferably, from 3 to 6, R represents a hydrocarbon radical, straight-chain or branched, with 1 to 4 carbon atoms, a halogen atom such as Cl or Br, or an alkoxy radical such as CH₃O—, x is equal to 0, 1, 2, 3 or 4 and when x=2, 3 or 4, the R radicals may be identical or different, which consists of reacting potassium phthalimide (II) with an α,ω-dibromoalkane (III) according to reaction (1):

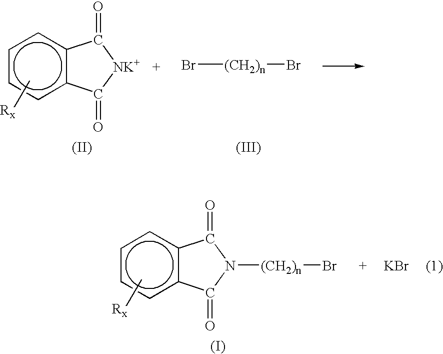

characterized in that it consists of carrying out the condensation reaction (1) of the potassium phthalimide (II) with the α,ω-dibromoalkane (III) without cosolvent, under stirring at a temperature from 50° C. to 130° C. and preferably, from 100 to 120° C., by using a molar ratio of α,ω-dibromoalkane (III) to potassium phthalimide (II) from 2.5 to 6 and, preferably from 3 to 4, then of removing the excess α,ω-dibromoalkane (III) under reduced pressure at a temperature equal to 150° C. at most, of taking up the reaction medium again with a low-boiling point alcohol at a temperature near the boiling point of said alcohol, of filtering at this temperature, of cooling the filtrate obtained and then of recovering the resulting crystallized product (I) by filtration followed by drying under reduced pressure at a temperature equal to 50° C. at most.

According to the present invention, an alcohol whose boiling point is equal to 100° C. at most is designated as the low boiling point alcohol.

Methanol, ethanol, isopropanol, n-propanol, tert-butanol, and 2-butanol will be mentioned by way of illustration of usable alcohols according to the present invention.

Preferably, ethanol or isopropanol will be used.

The condensation reaction (1) is carried out generally at atmospheric pressure, under an atmosphere of inert gas, such as nitrogen.

The reaction duration may vary to a great extent. The end of the reaction is determined by measurement of the KBr formed.

Generally, the reaction time is the time necessary to entirely transform the potassium phthalimide (II). It is a function of the stirring, the temperature and the molar ratio (III)/(II). The ratio influences the by production of products of dicondensation with formula:

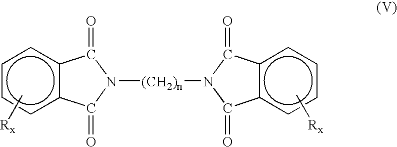

A molar ratio (III)/(II) less than 2.6 causes difficult stirring of the reaction medium and favors the formation of said products of dicondensation (V).

A molar ratio (III)/(II) greater than 6 does not provide any particular advantage and causes a decrease in productivity.

According to the present invention, the reaction medium, after removal of the excess α,ω-dibromoalkane, is taken up by a low boiling point alcohol which is brought to a temperature near its boiling point and the filtration is carried out at this temperature in order to remove the KBr formed and the possibly formed by-products that are not very soluble such as the product of dicondensation (V).

The procedure according to the present invention applies particularly well to the preparation of N-(ω-bromoalkyl)phthalimides with formula (I) in which x=0 and n is from 3 to 6.

The purity of the products obtained is sufficient to allow their use directly in later syntheses.

The examples that follow illustrate the invention.

### EXAMPLE 1

### Synthesis of N-(4-bromobutyl)phthalimide With a 1,4-dibromobutane/potassium Phthalimide Ratio of 3

Into a Sovirel type reactor mechanically stirred, provided with a condenser, an opening for introduction of solids, a system inerting with nitrogen, and a temperature probe, are loaded 655 g of 1,4-dibromobutane at 99% purity (3 mol) and 187 g of potassium phthalimide at 99% purity (1 mol). The reaction medium is brought under stirring between 115° C. and 120° C. and then kept at this temperature for 12 h. It is verified that the transformation of potassium phthalimide is completed by a measurement of the KBr in the reaction medium and then the excess 1,4-dibromobutane is evaporated under reduced pressure (10 to 18 mmHg). The recovery rate of the 1,4-dibromobutane is near 90% and this latter may be recycled directly into a later operation.

At the end of the distillation, the heterogeneous reaction medium is 120° C.-130° C. Its temperature is lowered to 70° C. and then 320 g of ethanol are introduced. The medium is kept at 70° C.

The contents of the reactor are filtered at 70° C. on a frit kept at this temperature. The moist cake is dried. It essentially contains KBr and diphthalimidobutane.

The ethanolic filtrate is left to cool to 20° C., which causes crystallization of the N-(4-bromobutyl)phthalimide. The ethanolic suspension of the product is filtered on the frit. The moist cake is washed with 2 ethanol fractions and then dried at 50° C. under reduced pressure (20 to 30 mmHg). Thus, 173 g of N-(4-bromobutyl)phthalimide are obtained or an isolated yield of 61% compared with the potassium phthalimide provided.

### EXAMPLES 2 TO 5

In these examples the operating protocol used in Example 1 is applied.

The reagents, their molar ratios, the general conditions and the results are collected in Table 1 below.

In this table:

P—K indicates potassium phthalimide

Rm represents the molar ratio Br(CH₂)ₙBr/P—K

Tx represents the recovery rate of the α,ω-dibromoalkane.

The molar yield is given in N-(ω-bromoalkyl)phthalimide isolated compared with the potassium phthalimide used.

TABLE 1

| | | | | | Conditions | | | | | |
|---|---|---|---|---|---|---|---|---|---|---|
| | Reagents | | | | | | | Purification | | |
| | Br(CH₂)ₙBr | | P-K | | T | Duration | Tx | | Quant. | Yield |
| Ex. | n | mole | mole | Rm | (° C.) | (h) | (%) | Alcohol | (g) | (%) |
| 2 | 4 | 3 | 1 | 3 | 115/120 | 12 | 89 | Isopropanol | 320 | 58 |
| 3 | 4 | 4 | 1 | 4 | 115/120 | 9 | 88 | Ethanol | 320 | 63 |
| 4 | 6 | 4 | 1 | 4 | 115/120 | 10 | 84 | Ethanol | 320 | 70 |
| 5 | 3 | 3.6 | 1 | 3.6 | 115/120 | 6 | 83 | Ethanol | 320 | 60 |

1. Procedure for preparation of N-(ω-bromoalkyl)phthalimides with formula: 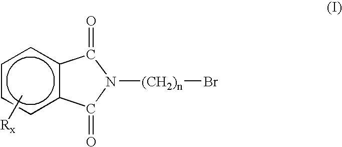
in which n is a whole number from 2 to 8, and preferably, from 3 to 6, R represents a hydrocarbon radical, straight-chain or branched, with 1 to 4 carbon atoms, a halogen atom such as Cl or Br, or an alkoxy radical such as CH₃O—, x is equal to 0, 1, 2, 3 or 4 and when x=2, 3 or 4, the R radicals may be identical or different, which consists of reacting potassium phthalimide (II) with an α,ω-dibromoalkane (III) according to reaction (1): 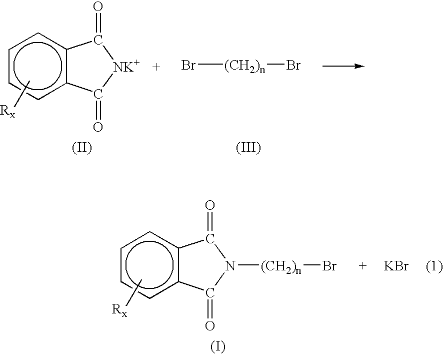
characterized in that it consists of carrying out the condensation reaction (1) of the potassium phthalimide (II) with the α,ω-dibromoalkane (III) without cosolvent, under stirring at a temperature from 50° C. to 130° C., by using a molar ratio of α,ω-dibromoalkane (III) to potassium phthalimide (II) from 2.5 to 6, then of removing the excess α,ω-dibromoalkane (III) under reduced pressure at a temperature equal to 150° C. at most, of taking up the reaction medium again with a low boiling point alcohol at a temperature near the boiling point of said alcohol, of filtering at this temperature, of cooling the filtrate obtained, and then of recovering the resulting crystallized product (I) by filtration followed by drying under reduced pressure at a temperature equal to 50° C. at most.

2. Procedure according to claim 1 characterized in that the temperature at which the condensation reaction (1) is carried out is from 100° C. to 120° C.

3. Procedure according to claim 1 or 2 characterized in that the molar ratio of α,ω-dibromoalkane (III) to potassium phthalimide (II) is from 3 to 4.

4. Procedure according to any one of claims 1 to 3 characterized in that the alcohol with low boiling point has a boiling point equal to 100° C. at most.

5. Procedure according to claim 4 characterized in that the alcohol with low boiling point is methanol, ethanol, isopropanol, n-propanol, tert-butanol or 2-butanol.

6. Procedure according to claim 5 characterized in that the alcohol with low boiling point is isopropanol or ethanol.

7. Procedure for preparation of N-(ω-bromoalkyl)phthalimides with formula (I) according to any one of claims 1 to 6 in which x=0 and n is from 3 to 6.

8. Procedure according to claim 7 in which n is equal to 4.
